(19) United States
(12) Patent Application Publication
(10) Pub. No.: US 2013/0302420 A1
(43) Pub. Date: Nov. 14, 2013

(54) OCULAR BIODEGRADABLE DRUG IMPLANT AND METHOD OF ITS USE

(71) Applicants: Barry L. Bowyer, Clearwater, FL (US); James P. Gills, Tarpon Springs, FL (US)

(72) Inventors: Barry L. Bowyer, Clearwater, FL (US); James P. Gills, Tarpon Springs, FL (US)

(21) Appl. No.: 13/470,427

(22) Filed: May 14, 2012

(51) Int. Cl. A61K 9/00; A61P 29/00; A61K 31/195

(52) U.S. Cl. 424/488; 514/567

(57) ABSTRACT

A biodegradable drug implant includes a PLG matrix and a non-steroid anti inflammation drug, for example diclofenac sodium. The implant is inserted into the eye in the anterior chamber, for example in the ciliary sulcus, following eye surgery. A method for treating and or preventing inflammation of the eye following eye surgery includes placing the implant in the anterior portion of the eye.

### BACKGROUND OF INVENTION

1. Field of the Invention

This invention is directed to a method of treating and/or preventing inflammation in the eye of a human, for example, following cataract surgery and to an implant composed of a slow release, biodegradable matrix impregnated with a non-steroid anti-inflammatory drug (NSAID) for use in the method of treating or preventing inflammation discussed above.

2. Description of Related Art

Inflammation of the eye often occurs following eye surgery. A well-known preventive treatment is to prescribe eye drops containing an effective amount of a non-steroid anti-inflammatory drug such as diclofenac sodium. For many patients use of eye drops is inconvenient and for elderly patients difficult to administer by themselves. Also this approach requires several applications of the eye drops over a period of time.

Recently there have been developments in biodegradable implants having steroid drugs in a matrix including polylactic and polyglycolic acid polymers in the formulation. See for example U.S. Pat. Nos. 8,034,370 and 7,048,946. These approaches have limited duration and have issues with the delivery rate of the drug.

Current approaches also used additional compounds such as release modulators, which are not necessary according to the present invention.

### BRIEF SUMMARY OF THE INVENTION

The present invention addresses the issues raised above with the development of a biodegradable implant having a matrix which includes a ratio of lactide to glycolide in the range of 90/10 to 80/20 by weight. Further the implant includes about 4-10 percent by weight of an NSAID such as diclofenac sodium.

The method of the invention includes the formulation of a solid implant prepared in accordance with the above parameters and placing the implant within the anterior chamber of the eye, for example in the ciliary sulcus region.

### BRIEF DESCRIPTION OF THE SEVERAL VIEWS OF THE DRAWING(S)

FIG. 1 is a schematic view showing various elements that make up the human eye.

FIG. 2 is a graph of the amount of diclofenac dissolved for several formulations versus time in days.

FIG. 3 is a graph of the amount of diclofenac dissolved for a formulation prepared according to an embodiment of the invention versus time in days.

### DETAILED DESCRIPTION OF THE INVENTION

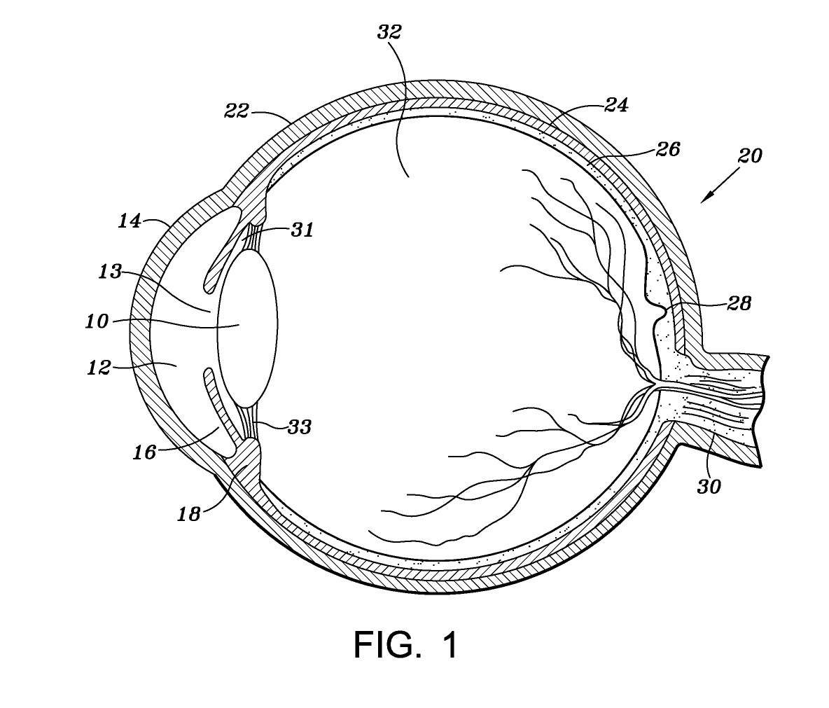

Referring to FIG. 1 it can be seen that the relevant portions of the eye for purposes of this invention are as follows. Eye 20 has a natural lens capsule 10 and pupil 13, which is surrounded by iris 16. The area forward of the lens capsule 10 is generally referred to as the anterior chamber 12 and the area rearward of the lens capsule 10 is referred to as the posterior chamber. The lens capsule 10 is held in place by ciliary zonules 33 which extend between the lens capsule 10 and the ciliary body 18. The space 31 between the iris 16 and the ciliary zonules 33 is referred to as the ciliary sulcus 31. The eye also includes cornea 14, sclera 22, choroid 24, retina 26, fovea 28, and optic nerve 30.

For cataract surgery, a small incision is normally made at the junction of cornea 14 and sclera 22 and the natural lens can be removed from the lens capsule by phacoemulsification, for example. An intraocular lens (IOL) is than implanted within the lens capsule through the same initial incision.

The formulation of this drug delivery device utilizes a polymer matrix of poly (DL-lactide-co-glycolide), commonly referred to as PLG. The ratio of lactide to glycolide may be changed in any proportion required to achieve the drug delivery characteristics necessary for the application and drug delivery site. The current formulations have ratios of (lactide:glycolide) which may include ratios from (90:10) to (80:20), lactide molecules to glycolide molecules, respectively. A preferred ratio for use with the non-steroidal drug diclofenac sodium is a ratio of (85:15). This ratio was chosen to give consistent release of the non-steroidal drug over a nine week period. Changing the ratio of (lactide:glycolide) alters the breakdown time of the polymer matrix and allows the duration of disintegration to be reduced or lengthened as needed.

The preparation of the drug delivery device requires the mixing of the matrix material, diclofenac sodium (or other drug), and a solvent, ethyl acetate. Depending upon the drug being mixed, other solvents such as acetone, acetonitrile, chloroform, dichloromethane, ethyl alcohol, and tetrahydrofuran may be employed. The preferred solvent for the diclofenac sodium preparation is ethyl acetate. It is considered a class 3 residual solvent from an FDA perspective, and therefore, is considered safe for use in pharmaceutical preparations within a specified residual limit.

Formulations comprising from a 4% drug loading to a 10% drug loading on a weight/weight basis may be used, depending upon the final concentration of drug desired for the device. An example of a formulation according to an embodiment of the invention is as follows.

A formulation comprising 94% by weight of matrix is mixed with 6% by weight of diclofenac sodium. The solvent is then added at 50% of the total weight of the matrix/drug mixture. The mixture is homogenized in a counter-rotational centrifugal mixer for a specified time period. After blending, the mixture is reweighed to determine how much of the solvent has been evaporated during the homogenization procedure. If there is more than 2% residual solvent remaining in the mixture, then the mixture is heated to 80° C. and placed back into the homogenizer for another cycle. This process is repeated as necessary to evaporate the solvent below the residual limits allowed for pharmaceutical preparations. Once completed, the material is heated and injected into molds containing preformed shapes (cylindrical rods, for example) which are then cut to size and weight. The devices are then packaged inside an implantation device such as a cartridge type delivery system, which is contained within a tyvek pouch. The tyvek pouch is heat sealed and boxed for terminal sterilization using gamma irradiation. After terminal sterilization, the drug delivery devices are ready for use.

The PLG matrix utilized in the above formulation can be obtained from Direct Corporation in Birmingham, Ala., under product number B60006-1 which is identified as 85:15 poly (DL-lactide-co-glycolide). The specifications for the product are DL-lactide: 80-90 mole %; glycolide 10-20 mole %.

Biodegradable implants prepared as described above were subjected to the following tests to determine the average amount of diclofenac dissolution over time.

Three types of implants (gamma sterilized) and containing different weight fractions of diclofenac (DF) were tested. The goal of this stage of the project was to assess the rate of DF dissolution as a function of DF concentration in the pellets, and to evaluate the effect of the sterilization on the dissolution rates and components.

To set up the experiment, three replicate implants were used for each provided formulation. Each implant was weighed, placed into sterile 10 ml tubes and 5 ml of balanced salt solution (BSS) was added to each tube. The experiment was conducted in semi-continuous solvent replacement mode. A 2 ml sample aliquot was taken from each tube daily for spectrophotometric measurements. The tubes were gently inverted a few times prior to the aliquot sampling to ensure homogeneous distribution of solutes in BSS. After sampling the solvent was decanted from the tubes (only ˜0.2 ml was left just enough to cover the pellet) and 5 ml of fresh BSS was carefully added to each tube. The tubes were kept stationary in a water bath at ˜37° C.

The UV-visible spectra were recorded using a diode array spectrometer (HP 8453 Hewlett-Packard, Palo Alto, Calif.). All measurements were conducted at room temperature using a 1 cm pathlength cuvette. Prior to recording a sample spectrum the spectrometer was zeroed to account for any stray light. To avoid the effect of inhomogeneities in the suspending medium, the background spectrum was taken using the same BSS utilized in the preparation of the samples.

The concentration of DF at each time point is determined from the UV-visible spectra in accordance with the Beer-Lambert law:

C=A(λ)/(l*ε(λ))

Where A(λ) denotes the measured optical density, l is the cuvette pathlength, ε(λ) is the DF extinction coefficient, and λ is the wavelength.

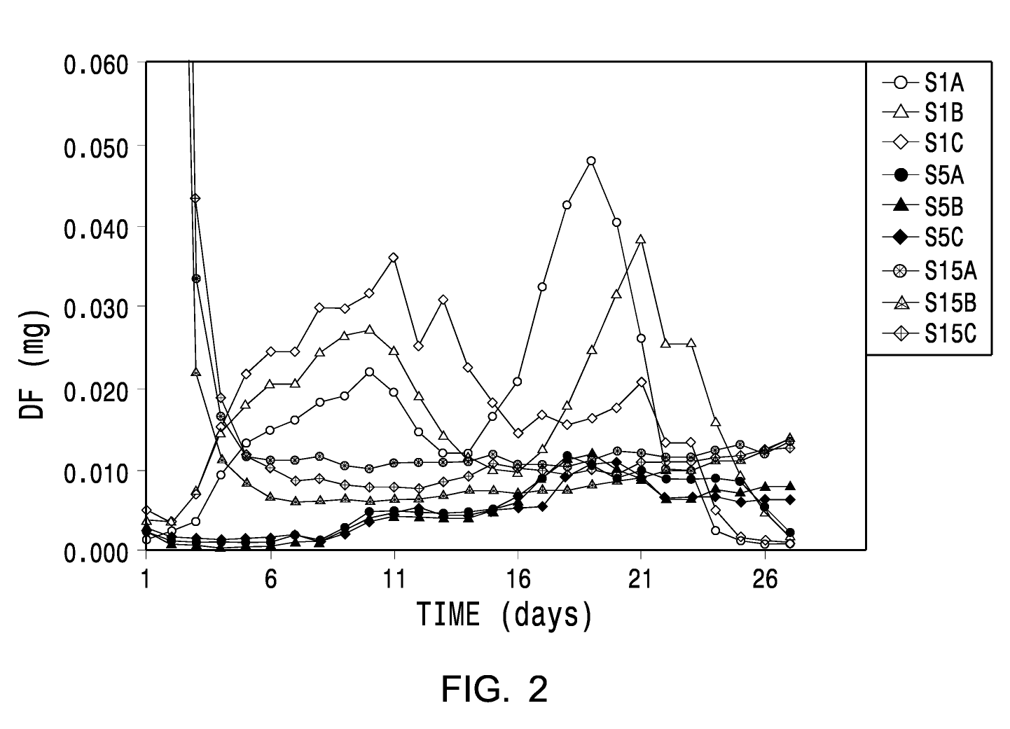

FIG. 2 shows multiple formulations which contain varying concentrations of diclofenac. Each formulation was run in triplicate to test the consistency of the desired drug loading levels with respect to the dissolution profile and ultimate delivery of the drug over time. The drug loading levels were tested at levels of 5%, and 15%, curve labels at S1, S5 and S15 denote the respective drug loading levels. A regression analysis was performed on the data which enabled predicted outcomes when varying concentration levels were entered into the resulting equation. The most consistent drug loading levels were found to be within the 4% to 10% range, based upon these experiments.

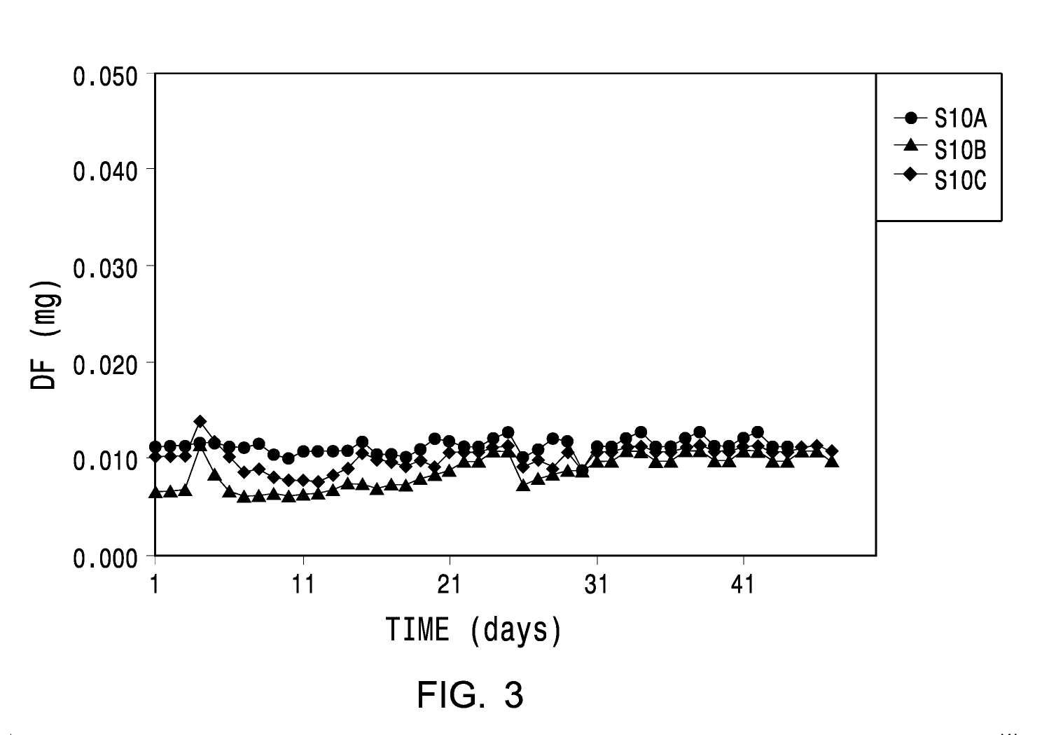

FIG. 3 shows tests results for the amount of diclofenac dissolved per day for a formulation prepared according to an embodiment of the invention.

Three implants were prepared using a 85:15 ratio of lactide:glycolide and a 10% drug loading by weight. Each implant weighed approximately 5 milligrams. As can be seen from the graph in FIG. 3, this formulation experienced a substantially linear dissolution rate of about 0.010 mg per day to beyond forty-one days.

Implants according to the invention are to be placed in the anterior chamber 12 of the eye and preferably in the ciliary sulcus 31 as shown in FIG. 1. This can be accomplished by dilating the pupil and passing by the iris.

This implant may take the form of a cylinder having a diameter from about 0.5 to 1.5 mm in diameter and about 2-4 mm in length. Other shapes are possible.

Placement in the vicinity of the surgically manipulated area within the eye is a more effective use of the drug being delivered. This technique allows less drug to be delivered directly to the site than would be required topically, due to poor absorption through the conical tissues. This reduces the possibility of unwanted systemic side effects, as well as reduces the costs associated with the drug itself. The current formulations appear to be effective in animal studies with as little as 2% of the topically administered (surface treated) drugs that are currently being used. Moreover, a sustained drug concentration is being delivered to the site over the course of treatment. No spikes of drug concentration are Observed as is found with repeated topical administration. In addition, the geriatric population has proven difficulty with self administration of topical drops due to visual problems, poor dexterity, and memory lapse.

Examples of non-steroid anti inflammation drugs that can be used include the following: Aspirin (Anacin, Ascription, Bayer, Bufferin, Ecotrin, Excedrin), Choline and magnesium salicylates, Choline salicylate, Celecoxib, Diclofenac potassium, Diclofenac sodium, Diclofenac sodium with misoprostol, Diflunisal, Etodolac, Fenoprofen calcium, Flurbiprofen, Ibuprofen, Indomethacin, Ketoprofen, Magnesium salicylate, Meclofenamate sodium, Mefenamic acid, Meloxicam, Nabumetone, Naproxen, Naproxen sodium, Oxaprozin, Piroxicam, Rofecoxib, Salsalate, Sodium salicylate, Sulindac, Tolmetin sodium, and Valdecoxib.

I claim:

1. A biodegradable drug implant for treating and or preventing inflammation in the eye following eye surgery comprising:
a matrix for slowly releasing a drug, the matrix comprising poly (DL-lactide-co-glycolide) wherein the ratio of lactide to glycolide is in the range of 80/20 to 90/10 by weight; and
a non-steroid anti-inflammation drug (NSAID) comprising about four to about ten percent of the total weight of the implant dispersed within the matrix.

2. An implant according to claim 1 wherein the NSAID comprises diclofenac.

3. An implant according to claim 1 wherein the NSAID is selected from the group consisting of Aspirin (Anacin, Ascription, Bayer, Bufferin, Ecotrin, Excedrin), Choline and magnesium salicylates, Choline salicylate, Celecoxib, Diclofenac potassium, Diclofenac sodium, Diclofenac sodium with misoprostol, Diflunisal, Etodolac, Fenoprofen calcium, Flurbiprofen, Ibuprofen, Indomethacin, Ketoprofen, Magnesium salicylate, Meclofenamate sodium, Mefenamic acid, Meloxicam, Nabumetone, Naproxen, Naproxen sodium, Oxaprozin, Piroxicam, Rofecoxib, Salsalate, Sodium salicylate, Sulindac, Tolmetin sodium, and Valdecoxib.

4. A method of treating and/or preventing inflammation in the eye following eye surgery comprising:
placing a biodegradable drug implant comprising a polymer matrix and a NSAID drug in an implantation device having an outlet,
positioning the outlet of the implantation device within the anterior portion of the eye following eye surgery, and
operating the implantation device so as to position the biodegradable drug implant within the anterior portion of the eye.

5. The method of claim 4 wherein the outlet of the implantation device is positioned within the ciliary sulcus portion of the anterior chamber and the implant is thereby positioned within the ciliary sulcus.

6. The method of claim 4 wherein the biodegradable drug implant comprises:
a matrix for slowly releasing a drug, the matrix comprising poly (DL-lactide-co-glycolide) wherein the ratio of lactide to glycolide is in the range of 80/20 to 90/10 by weight; and
a non-steroid anti-inflammation drug (NSAID) comprising about four to about ten percent of the total weight of the implant dispersed within the matrix.

7. The method of claim 4 wherein the NSAID is diclofenac.

8. The method of claim 7 wherein the NSAID is selected from the group consisting of Aspirin (Anacin, Ascription, Bayer, Bufferin, Ecotrin, Excedrin), Choline and magnesium salicylates, Choline salicylate, Celecoxib, Diclofenac potassium, Diclofenac sodium, Diclofenac sodium with misoprostol, Diflunisal, Etodolac, Fenoprofen calcium, Flurbiprofen, Ibuprofen, Indomethacin, Ketoprofen, Magnesium salicylate, Meclofenamate sodium, Mefenamic acid, Meloxicam, Nabumetone, Naproxen, Naproxen sodium, Oxaprozin, Piroxicam, Rofecoxib, Salsalate, Sodium salicylate, Sulindac, Tolmetin sodium, and Valdecoxib.

9. The method of claim 4 wherein the eye surgery is cataract surgery.
The implant as claimed in claim 1, wherein the implant is in the shape of a cylinder having a diameter in the range of about 0.5 mm to 1.5 mm and a length of about 2 to 4 mm.
The method of claim 4 wherein in implant is the shape of a cylinder having a diameter in the range of about 0.5 mm to 1.5 mm and a length of about 2 to 4 mm.
